(19) United States
(12) Patent Application Publication
(10) Pub. No.: US 2008/0139434 A1
(43) Pub. Date: Jun. 12, 2008

(54) Concentrated surfactant compositions

(75) Inventors: Geetha Basappa, Rotterdam (NL); Maria Teresa Belmar, Sharnbrook (GB); Julia Helen Telford, Sharnbrook (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc, d/b/a UNILEVER

(21) Appl. No.: 11/999,788

(22) Filed: Dec. 7, 2007

(30) Foreign Application Priority Data
Dec. 8, 2006 (EP) EP06125743

(51) Int. Cl. A61K 8/46

(52) U.S. Cl. 510/159

(57) ABSTRACT

The present invention relates to a composition comprising a high amount of a specific surfactant mixture and water, as well as a method of manufacture thereof.

The present invention relates to a composition comprising a high amount of a specific surfactant mixture and water, as well as to a method of manufacture thereof.

Concentrated formulations comprising surfactants as well as mixtures of surfactants are usually provided to reduce the transport costs as well as to reduce storage capacities Usually the commonly known concentrates comprise all essential ingredients for producing, for example, a personal care composition with the exception of solvent (which is usually water).

The goal of the present invention is to provide a highly concentrated surfactant composition which is stable, which can serve as a base composition (skeleton composition) and which can be handled reasonably easily and comprises two surfactants and water only.

FR 2 826 017 A1 discloses a mixture of surfactants comprising an alkyl and/or alkenyl oligoglycoside, betaine and an alkyl ether sulphate

EP 1 225 214 A2 discloses a detergent composition which foams well and is not slimy containing an alkali metal taurate salt or an organic alkali taurate salt of N-acylmethyltaurate, N-acyltaurine, alkylsulphuric ester, alkyl ether sulphuric ester or alkylsulphonic acid.

DE 199 44 544 A1 discloses a surfactant mixture comprising an alkyloligoglycoside and an alkyl- and/or alkenylether sulphate.

FR 2 398 797 A1 discloses concentrated aqueous surfactant compositions comprising mixtures of different surfactants for use in aqueous systems.

WO 99/09944 A1 discloses aqueous nacreous lustre dispersions containing fatty acid polyglycol ester sulphate, emulsifiers, nacreous lustre waxes and polyols.

DE 196 41 280 A1 discloses waterborne pearlescent dispersions comprising monoglyceride (ether)sulphate, emulsifiers and polyols.

EP 0 922 754 A1 discloses pearlescent compositions comprising a fatty acid, an alkylether sulphate or acylisethionate, and water.

DE 196 46 882 A1 discloses an aqueous polyol free pearlescing concentrate based on selected surface-active emulsifiers and pearlescent waxes

### DESCRIPTION OF THE INVENTION

The present invention relates to a composition comprising:

(i) 50-95% by weight, based on the total weight of the composition, of
(ia) an alkyl ether sulphate and/or salt thereof, and
(ib) at least one further surfactant which is an anionic and/or amphoteric surfactant, and
(ic) optionally one or more non-ionic surfactants, and

(ii) 5-50% by weight, based on the total weight of the composition, of water, wherein the alkyl ether sulphate and/or salt thereof of (ia) and the anionic surfactant of (ib) are not identical surfactants

Preferred compositions comprise 50-90%, preferably 60-95%, more preferably 60-95%, most preferably 60-85% by weight of (i). As a consequence thereof preferred compositions comprise 10-50%, preferably 5-40%, more preferably 10-40%, most preferably 15-40% by weight, based on the total weight of the composition, of water.

A further embodiment of the present invention is a composition where the ratio of (ia):[(ib)+(ic)] is 1:2 to 40:1, preferably 1:1 to 30:1.

Preferred compositions comprise ≦10% by weight, based on the total amount of the composition, of non-ionic surfactants.

The compositions according to the present invention have the advantage that without using any further additives, the compositions show good stability as well as good handling properties. Without the need to include any further additives (such as, for example, preservatives), it opens a wide field of interesting applications and uses. A user can, for example, choose to add a preservative (or other ingredients) or not. The user can be found in industry as well as in private households

The composition according to the present invention is preferably used as a base composition for a personal care composition. Preferred personal care compositions are body washes (such as for shower or bath) and/or shampoos.

The compositions according to the present invention preferably have a pH≧5, preferably ≧5.5.

The composition according to the present invention preferably excludes added preservative, thus the composition may contain traces of preservatives which can be found in the commercially available surfactants and which cannot be removed completely. The amount of preservative in the composition is usually less than 0.1% by weight of the composition.

The term preservative is to be understood as a natural or synthetic chemical that is added to products to retard spoilage, whether from microbial growth, or undesirable chemical changes. A distinction is sometimes made between anti-microbial preservatives which function by inhibiting the growth of bacteria and fungi, and antioxidants such as oxygen absorbers, which inhibit the oxidation of food constituents. Common anti-microbial preservatives include sodium nitrate, sodium nitrite, sulphites (sulfur dioxide, sodium bisulfite, potassium hydrogen sulfite, etc.) and disodium EDTA. Antioxidants include BHA and BHT. Other preservatives include formaldehyde (usually in solution), glutaraldehyde, diatomaceous earth (kills insects), ethanol, dimethyl dicarbonate and methylchloroisothiazolinone. Further preservatives include 2-bromo-2-nitropropane-1,3-diol, phenoxy ethanol citric acid, tartaric acid, phosphoric acid, iminodiacetic acid, nitrilotriacetic acid, hydroxyethyleneaminodiacetic acid and ethylenediaminetetraacetic acid and salts thereof, para-hydroxybenzoates such as butyl paraben, methyl paraben and propyl paraben; imidazolines (e.g. imidiazolinylurea), triclosan, hydantoins (e.g. dimethyloldimethylhydantoin), isothiazolidinone compounds; and mixtures thereof. Commercially available preservatives include Kathon® CG, Kathon® CGII and Myacide®.

A further preferred embodiment of the present invention relates to a composition excluding a pearlescent agent or an opacifying agent. Examples of a pearlescent or an opacifying agent include, but are not limited to: mono- or diesters of (a) fatty acids having 16 to 22 carbon atoms and (b) either ethylene or propylene glycol; mono- or diesters of (a) fatty acids having 16 to 22 carbon atoms (b) a polyalkylene glycol of the formula HO-(JO)ₐ-H, wherein J is an alkylene group having 2 to 3 carbon atoms, and a is 2 or 3, and fatty alcohols containing 16 to 22 carbon atoms; fatty esters of the formula KCOOCH₂L, wherein K and L independently contain 15 to 21 carbon atoms; inorganic solids insoluble in the composition, and mixtures thereof.

The compositions according to the present invention always comprise alkyl ether sulphates or salts thereof. Preferred alkyl ether sulphates salts are those of formula

RO—(CH₂CH₂O)ₓSO₃M,

wherein R is an alkyl or alkenyl group of 8 to 24 carbon atoms,

x is an integer having a value of 1 to 10, and

M is a cation such as ammonium, alkanolamines such as triethanolamine, monovalent metals such as sodium and potassium, and polyvalent metal cations such as magnesium and calcium.

Preferably, R has 8 to 18 carbon atoms, more preferably 10 to 16 carbon atoms, even more preferably 12 to 14 carbon atoms. The alkyl ether sulphate salts are typically made as condensation products of ethylene oxide and monohydric alcohols having 8 to 24 carbon atoms The alcohols can be synthetic or they can be derived from fats, e.g. coconut oil, palm kernel oil and tallow. Lauryl alcohol and other straight chain alcohols derived from coconut oil or palm kernel oil are preferred. Such alcohols are reacted with between 0 and 10, preferably 1 to 5, more preferably 2 to 3, molar proportions of ethylene oxide, and the resulting mixture of molecular species having, for example, an average of 3 moles of ethylene oxide per mole of alcohol, is sulphated and neutralized.

Specific non-limiting examples of alkyl ether sulphate salts which may be used in the compositions of the present invention include sodium and ammonium salts of coconut alkyl triethylene glycol ether sulphate, tallow alkyl diethylene glycol ether sulphate, tallow alkyl triethylene glycol ether sulphate and tallow alkyl hexaethylene sulphate. The preferred alkyl ether sulphate salt is sodium laureth sulphate.

As component (ib) at least one anionic and/or at least one amphoteric surfactant is present The anionic surfactant may be selected from the group consisting of alkyl ether sulphate, alkyl sulphates, ethoxylated alkyl sulphates, alkyl ethoxycarboxylates, methyl acyl taurates, fatty acyl glycinates, N-acyl glutamates, acyl isethionates, alkyl sulfosuccinates, alkyl ethoxysulphosuccinates, alpha sulphonated fatty acids, esters of alpha sulphonated fatty acids, alkyl phosphate esters, ethoxylated alkyl phosphate esters, acyl sarcosinates, acyl aspartates, alkoxy cocamide carboxylates, (ethoxylated) alkanolamide sulphosuccinates, ethoxylated alkyl citrate sulphosuccinates, acyl ethylene diamine triacetates, acylhydroxyethyl isethionates, acyl amide alkoxy sulphates, linear alkyl benzene sulphonates, paraffin sulphonates, alpha olefin sulphonates, fatty acid/protein condensates, salts thereof, soaps such as ammonium, magnesium, potassium, triethanolamine and sodium salts of lauric acid, myristic acid and palmitic acid, and mixtures thereof. Alkyl and/or acyl chain lengths for these surfactants are C₆-C₂₂, preferably C₁₂-C₁₈ more preferably C₁₂-C₁₄

Amphoteric surfactants suitable for use in the composition are well known in the art, and include those surfactants broadly described as derivatives of an aliphatic quaternary ammonium, phosphonium, or sulphonium compound, in which the aliphatic radicals can be straight or branched chain, and wherein one aliphatic substituent contains 8 to 18 carbon atoms and the same or different aliphatic substituent contains an anionic group such as a carboxy, a sulphonate, a sulphate, a phosphate or a phosphonate group. Zwitterionics such as betaines are preferred A particularly preferred betaine is cocamidopropyl betaine

The non-ionic surfactant as component (ic) can be chosen from any commonly known non-ionic surfactant, such as alkyl poly(ethylene oxide) alkyl polyglucosides (such as octyl glucoside and decyl maltoside), fatty alcohols (such as cetyl alcohol and oleyl alcohol), cocamide MEA, cocamide DEA and cocamide TEA.

The composition according to the present invention may be used in a personal care composition. Preferably the personal care compositions are body washes (for shower and bath) and shampoos. The personal care composition is produced by dilution and addition of optional further ingredients. If further ingredients (additives) are necessary in the personal care composition it is also possible to add them before and/or during and/or after the dilution process. The composition according to the present invention can comprise one or more additive. Such additives can include cationic polymers, particles, conditioning agents (hydrocarbon oils, fatty esters, silicones), anti-dandruff agents, suspending agents, viscosity modifiers, dyes, non-volatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers and vitamins. When a composition according to the present invention comprises at least one of these ingredients, then the amount is in the range 1 to 20% by weight, based on the total weight of the composition. The amount is dependent on the degree of dilution.

Preferably a composition according to the present invention is translucent.

For ease of handling (e g pouring out of a container or pumping through pipes) it is important that the composition as described above is formulated such that it forms a liquid crystalline lamellar and/or lamellar gel phase and thus not a hexagonal phase

In another aspect of the invention, a process for manufacturing a composition according to the invention is provided, the process comprising the steps of:
(a) providing the alkyl ether sulphate or salt thereof in the form of a mixture with no more than 35% by weight, based on the total weight of the mixture, of water; then
(b) adding to and mixing with the mixture the at least one further surfactant chosen from the group consisting of anionic and/or amphoteric surfactants in substantially anhydrous form and the optional one or more non-ionic surfactants in substantially anhydrous form thereby to form the composition.

By substantially anhydrous is meant less than 10%, preferably less than 5%, more preferably less than 1% and most preferably less than 0.1% by weight, based on the weight of the surfactant, of water

By manufacturing the compositions of the invention in this manner, the alkyl ether sulphate or salt thereof always remains in a liquid crystalline lamellar phase or lamellar gel phase throughout the process. The advantage of this is that the viscosity of the composition always remains at a level where the handling and processing of the composition is easier.. If the percentage amount of water is allowed to rise, for example through addition of an aqueous solution of the anionic and/or amphoteric surfactants or the optional one or more non-ionic surfactants, the composition will enter the hexagonal phase which has been observed to have significantly higher viscosities such that handling and processing of the composition becomes much more difficult. The various phases may be identified using optical microscopy

Preferably the mixture of alkyl ether sulphate or salt thereof and water comprises no more than 30%, preferably no more than 25% and more preferably no more than 20% by weight, based on the total weight of the mixture, of water.

In another aspect of the invention, a process for manufacturing a personal care composition is provided comprising the steps of.
(a) providing a composition according to the invention; then
(b) diluting the composition with water using a high shear in-line mixer thereby to arrive at the personal care composition

Preferably the personal care composition is a body wash or a shampoo.

A high shear in-line mixer is required in the preparation of a personal care composition from a composition of the invention because in diluting the composition with water, the composition will pass through the highly viscous hexagonal phase before entering the low viscosity micellar phase.

### BRIEF DESCRIPTION OF THE FIGURE

FIG. 1 shows a schematic diagram of dilution with water of the composition of the invention to form a personal care composition.

The present invention will now be described further with reference to the following non-limiting examples. The percentages are all weight percentages and the temperatures are given in Celsius if not otherwise stated.

### EXAMPLE 1-7

### Compositions

The following compositions have been produced by using high shear mixing methods to incorporate the amphoteric surfactant into the mix. For some of the formulations such as those containing CMEA the mixtures are heated to 60° C. and then subjected to high shear mixing. After these mixtures are homogenised they are cooled to room temperature under shear and then transferred to a container.

TABLE 1
Compositions

| | Sodium | | | | |
|---|---|---|---|---|---|
| | laureth | | Cocomono | | |
| | sulfate | Cocamidopropyl | ethanolamide | Water | |
| | (SLES) | betaine (CAPB) | (CMEA) | [weight | |
| Example | [weight (%)] | [weight (%)] | [weight (%)] | (%)] | pH |
| 1 | 66.1 | 6 | — | 27.9 | 6.5 |
| 2 | 62.5 | 11.4 | — | 26.1 | 6.2 |
| 3 | 59.3 | 16.2 | — | 24.5 | 6.3 |
| 4 | 56.4 | 20.5 | — | 23.1 | 6 |
| 5 | 41.8 | 41.8 | — | 16.4 | 5.8 |
| 6 | 38 | 45.6 | — | 16.4 | — |
| 7 | 63.3 | — | 9.2 | 27.2 | 7.6 |

### EXAMPLES 8-9

### Personal Care Compositions

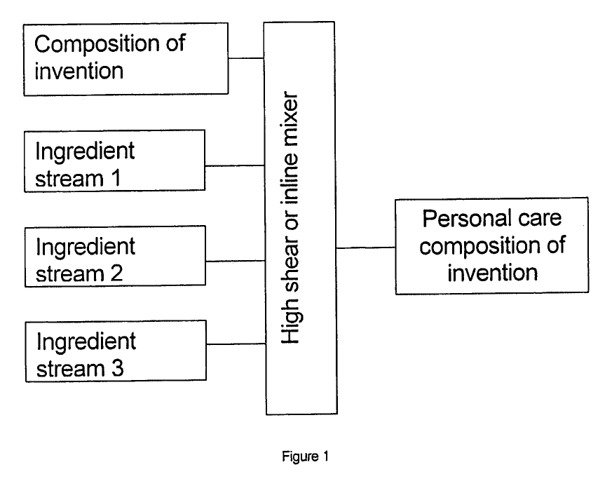

The following shower gels have been produced by making up the various other ingredients present in the shower gel into liquid ingredient streams. In some cases the liquid stream can also be pure water. These ingredient streams and the composition according to the present invention are then combined together using a high shear in-line mixing process (sonolator or Silverson mixer) to arrive at a homogeneous shower gel. It is important to use a high shear inline mixer to handle the highly viscous hexagonal phase that is produced during the dilution process. The homogenisation of the hexagonal phase is difficult to handle with conventional mixing processes. This process in which the product is prepared by mixing various liquid containing streams to the composition of the invention also enables the shower gels to be customised or personalised by tuning the level and number of the individual ingredient streams that are added to the product. A schematic diagram for the dilution process is shown in FIG. 1.

### Example 8

### Shower Gel

| Ingredient | Weight (%) |
|---|---|
| Concentrate according to the present invention | 15.50 |
| (composition of example 2) | |
| Carbopol Aqua SF-1 (30% active) | 4.00 |
| EDTA NA | 0.10 |
| NaOH | 0.50 |
| Formalin @37% | 0.14 |
| Vacuum salt | 1.60 |
| Citric Acid | 0.14 |
| Perfume | 0.70 |
| Water | 74.64 |

### Example 9

### Shower Gel

| Ingredient | Weight (%) |
|---|---|
| Concentrate according to the present invention | 16.24 |
| (composition of example 2) | |
| Carbopol Aqua SF-1 | 5.00 |
| DMDM Hydantoin | 0.20 |
| NaOH (100%) | 0.11 |
| Citric Acid Monohydrate | 0.18 |
| Fragrance | 1.00 |
| Acusol OP302 | 1.50 |
| NaCl (100%) | 0.78 |
| Jaguar C13S | 0.15 |
| Water | 69.93 |

1. A composition comprising:
(i) 50-95% by weight, based on the total weight of the composition, of
(ia) an alkyl ether sulphate and/or salt thereof, and
(ib) at least one further surfactant which is an anionic and/or amphoteric surfactant, and
(ic) optionally one or more non-ionic surfactants, and (ii) 5-50% by weight, based on the total weight of the composition, of water, wherein the alkyl ether sulphate and/or salt thereof of (ia) and the anionic surfactant of (ib) are not identical surfactants.

2. A composition according to claim 1 comprising 50-90%, by weight of (i).

3. A composition according to claim 1 wherein the ratio of (ia):[(ib)+(ic)] is 1:2 to 40:1.

4. A composition according to claim 1 comprising no more than 10% by weight of (ic).

5. A composition according to claim 1 having a pH≧5.

6. A composition according to claim 1 excluding added preservative.

7. A composition according to claim 1 excluding a pearlescent agent and/or an opacifying agent.

8. A composition according to claim 1 wherein (ia) is an alkyl ether sulphate salt of formula (I)
RO—(CH₂CH₂O)ₓSO₃M (I)
wherein R is an alkyl or alkenyl group of 8 to 24 carbon atoms,
x is an integer having a value of 1 to 10, and
M is a cation such as ammonium, alkanolamines such as triethanolamine, monovalent metals such as sodium and potassium, and polyvalent metal cations such as magnesium and calcium.

9. A composition according to claim 8 wherein (ia) is an alkyl ether sulphate salt selected from the group consisting of sodium and ammonium salts of coconut alkyl triethylene glycol ether sulphate, tallow alkyl diethylene glycol ether sulphate, tallow alkyl triethylene glycol ether sulphate and tallow alkyl hexaethylene sulphate.

10. A composition according to claim 9 wherein (ia) is sodium laureth sulphate.

11. A composition according to claim 1 wherein (ib) is an anionic surfactant selected from the group consisting of alkyl ether sulphate, alkyl sulphates, ethoxylated alkyl sulphates, alkyl ethoxycarboxylates, methyl acyl taurates, fatty acyl glycinates, N-acyl glutamates, acyl isethionates, alkyl sulphosuccinates, alkyl ethoxysulphosuccinates, alpha sulphonated fatty acids, esters of alpha sulphonated fatty acids, alkyl phosphate esters, ethoxylated alkyl phosphate esters, acyl sarcosinates, acyl aspartates, alkoxy cocamide carboxylates, (ethoxylated)alkanolamide sulphosuccinates, ethoxylated alkyl citrate sulphosuccinates, acyl ethylene diamine triacetates, acylhydroxyethyl isethionates, acyl amide alkoxy sulphates, linear alkyl benzene sulphonates, paraffin sulphonates, alpha olefin sulphonates, fatty acid/protein condensates, salts thereof, soaps such as ammonium, magnesium, potassium, triethanolamine and sodium salts of lauric acid, myristic acid and palmitic acid, and mixtures thereof and/or
an amphoteric surfactant which is a derivative of an aliphatic quaternary ammonium, phosphonium, or sulphonium compound, in which the aliphatic radicals can be straight or branched chain, and wherein one aliphatic substituent contains 8 to 18 carbon atoms and the same or different aliphatic substituent contains an anionic group such as a carboxy, a sulphonate, a sulphate, a phosphate or a phosphonate group.

12. A composition according to claim 11 wherein (ib) is cocamidopropyl betaine.

13. A composition according to claim 1 wherein (ic) is a non-ionic surfactant selected from the group consisting of alkyl poly(ethylene oxide) alkyl polyglucosides such as octyl glucoside and decyl maltoside, fatty alcohols such as cetyl alcohol and oleyl alcohol, cocamide MEA, cocamide DEA and cocamide TEA.

14. A composition according to claim 1 comprising at least one further additive.

15. A composition according to claim 1 wherein the composition forms a liquid crystalline lamellar phase or lamellar gel phase.

16. A process for manufacturing a composition according to claim 1, the process comprising the steps of:
(a) providing the alkyl ether sulphate or salt thereof in the form of a mixture with no more than 35% by weight, based on the total weight of the mixture, of water; then
(b) adding to and mixing with the mixture the at least one further surfactant chosen from the group consisting of anionic and/or amphoteric surfactants in substantially anhydrous form and the optional one or more non-ionic surfactants in substantially anhydrous form thereby to form the composition.

17. A process for manufacturing a composition according to claim 16 wherein the mixture of alkyl ether sulphate or salt thereof and water comprises no more than 30%, by weight, based on the total weight of the mixture, of water.

18. A process for manufacturing a composition according to claim 16 wherein the alkyl ether sulphate or salt thereof remains in a liquid crystalline lamellar phase or lamellar gel phase throughout the process.

19. A process for manufacturing a personal care composition comprising the steps of:
(a) providing a composition according to claim 1; then
(b) diluting the composition with water using a high shear in-line mixer thereby to arrive at the personal care composition.

20. A process for manufacturing a personal care composition wherein the personal care composition is a body wash or a shampoo.
